(19) United States
(12) Patent Application Publication
(10) Pub. No.: US 2026/0315293 A1
(43) Pub. Date: Oct. 15, 2026

(54) ABSORBENT ARTICLES INCLUDING A WATER-RESPONSIVE FILM AND METHODS OF PRODUCING THE SAME

(71) Applicant: Kimberly-Clark Worldwide, Inc., Nennah, WI (US)

(72) Inventors: Richmond R. Cohen, Appleton, WI (US); Ingrid C. Hoeger Vivas, Chamblee, GA (US); Uyen T. Lam, Menasha, WI (US); Wing-Chak Ng, Roswell, GA (US); Peiguang Zhou, Suwanee, GA (US)

(73) Assignee: Kimberly-Clark Worldwide, Inc., Nennah, WI (US)

(21) Appl. No.: 18/998,833

(22) PCT Filed: Jul. 13, 2023
(86) PCT No.: PCT/US2023/027685

§ 371 Date: Jan. 27, 2025

Related U.S. Application Data
(60) Provisional application No. 63/400,645, filed on Aug. 24, 2022.

(51) Int. Cl. A61F 13/539; A61F 13/00; A61F 13/15; A61F 13/511; A61F 13/512; A61F 13/514; A61L 15/22; A61L 15/44; A61L 15/48; A61L 15/52; A61L 15/60

(52) U.S. Cl. CPC A61F 13/539; A61F 13/00063; A61F 13/15723; A61F 13/51121; A61F 13/5123; A61F 13/51405; A61L 15/225; A61L 15/44; A61L 15/48; A61L 15/52; A61L 15/60; A61F 2013/51147

(57) ABSTRACT

The present disclosure relates to absorbent articles including a water-responsive film. The film includes a crystalline water-dispersible polymer and an aromatic thermoplastic polyurethane elastomeric polymer. The film has a crystallinity of at least 25% and up to 60%. The film may include one or more apertures that can transition between an open and closed configuration in response to water, effecting the intake and rewet attributes when used in an absorbent article. The film expands when in contact with water, but retracts as it dries, allowing for multiple insults to trigger the expansion and retraction properties of the film.

### CROSS-REFERENCE TO RELATED APPLICATION

This application claims the benefit of and priority to U.S. Provisional Patent Application No. 63/400,645, filed Aug. 24, 2022, which is hereby incorporated by reference in its entirety for all intents and purposes.

### FIELD

The present disclosure generally relates to absorbent articles. More particularly, the present disclosure relates to absorbent articles including a water-responsive film that expands when in contact with water (e.g., from bodily fluid) to close one or more apertures that initially facilitates passage of a fluid.

### BACKGROUND

Absorbent articles such as diapers, incontinence garments, sanitary napkins, and menstrual pads, are designed to absorb and retain liquid and other discharges from the human body to prevent soiling of the body and clothing. These articles absorb and contain fluids (e.g., bodily waste) and are intended to be discarded after a limited period of use. Conventional absorbent articles comprise an absorbent body disposed between an inner layer adapted for contacting the wearer's skin and permitting passage of fluid into the absorbent body and an outer layer for inhibiting leakage of the absorbed liquid out of the article. The inner layer of the absorbent article is typically liquid permeable to permit body waste to pass therethrough for absorption by the absorbent body.

A number of absorbent articles that collect fluids are commercially available. However, these absorbent articles have a tendency to leak before the fluid absorbent capacity is entirely used. For example, an absorbent structure of an absorbent article may have a fluid intake rate determined by the structure and materials of the absorbent structure. If the fluid flow to the absorbent structure exceeds the fluid uptake rate, the fluid may leak onto the surface of the absorbent article. This can cause direct contact between fluid and a user's skin, which can result in overhydration of the skin, rendering it susceptible to irritation and infection.

Additionally, absorbent articles should provide rapid passage of fluid (e.g., urine or menses) into the absorbent structure of the absorbent article and should retain the fluid therein. If the fluid is not adequately retained within the absorbent structure of the absorbent article, pressure applied to the absorbent article may cause the fluid to travel from the interior of the absorbent article through a surface of the absorbent article. To provide rapid intake of fluid, the materials comprising the absorbent structure of the absorbent article need to have sufficient permeability and wettability to facilitate intake of the fluid. Although these properties of absorbent articles provide for rapid intake of fluid, they also allow for easy flowback, i.e., passage of fluid out of the absorbent article and onto the user's skin, when pressure is applied to the absorbent article. As a result, there is a correlation between the rate of fluid intake and the rate and volume of flowback in conventional absorbent articles, giving rise to a phenomenon known as “easy in, easy out.” Accordingly, absorbent articles that are capable of rapid intake of fluid often have greater flowback of fluid, and thus higher levels of skin overhydration. Conversely, absorbent articles that exhibit limited flowback of fluid, and thus less leakage, tend to have slow fluid intake and thus more leakage.

In addition to problems with leakage in some absorbent articles, there are also hygienic issues that directly affect the user. Often the body fluid that leaks to the surface of the absorbent article sits in direct contact with the user which makes for an unpleasant and unclean feel. Particularly with feminine hygiene products such as sanitary napkins, the unpleasant or unclean feeling may lead to poor perception of product performance and the inability to get maximum use from the product.

There remains a need for an absorbent article that can adequately reduce the incidence of leakage of fluid from the absorbent article while supporting rapid intake of fluid.

### SUMMARY

Covered embodiments of the present disclosure are defined by the claims, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

In some embodiments, the present disclosure is directed to an absorbent article. The absorbent article includes a film comprising a water-soluble polymer and an aromatic thermoplastic polyurethane, wherein the film has a degree of crystallinity greater than or equal to 25%, wherein the film is configured to expand when in contact with a fluid. In some embodiments, the water-soluble polymer comprises polyethylene oxide, hydroxypropyl cellulose, polyvinyl alcohol, polyvinyl pyrrolidone, polyvinyl pyridine, gelatinized starch, nylon copolymers, polyacrylic acid, or combinations thereof. In some embodiments, wherein the film includes a patterned surface including one or more apertures, wherein the apertures comprise at least 10% of the total surface area of the film. In some embodiments, a region adjacent the one or more apertures is treated with a hydrophilic agent. In some embodiments, a portion of the film is bonded to a substrate. In some embodiments, the region of the film adjacent the one or more apertures is not bonded to a substrate. In some embodiments, the aromatic thermoplastic polyurethane comprises a polyester-based thermoplastic polyurethane. In some embodiments, the film has a degree of crystallinity from 25% to 60%. In some embodiments, the film comprises the water-soluble polymer in an amount from 10 wt. % to 90 wt. %, based on the total weight of the film. In some embodiments, the film comprises the aromatic thermoplastic polyurethane in an amount from 10 wt. % to 90 wt. %, based on the total weight of the film. In some embodiments, a weight ratio of water-soluble polymer to the aromatic thermoplastic polyurethane is at least 1:1. In some embodiments, the film comprises the water-soluble polymer in an amount from 30 wt. % to 70 wt. %, based on the total weight of the film, thermoplastic polyurethane in an amount from 30 wt. % to 60 wt. %, based on the total weight of the film, and a ratio of ratio of water-soluble polymer to aromatic thermoplastic polyurethane is at least 1.5:1. In some embodiments, the film comprises one or more additives comprising surfactant, absorbents, antibiotics, or skin benefit agents. In some embodiments, the one or more additives comprise a superabsorbent. In some embodiments, the absorbent article includes a fluid transfer layer between the film and an absorbent body. In some embodiments, the film further comprises a 3-D structure treated with a hydrophobic agent.

In some embodiments, the present disclosure provides an absorbent article including: a film comprising a water-soluble polymer and an aromatic thermoplastic polyurethane, wherein the film has a degree of crystallinity greater than or equal to 25%; a substrate bonded to the film to produce a laminate; a backsheet; and an absorbent body between the laminate and the backsheet.

In some embodiments, the present disclosure provides a method of producing a laminate. The method includes providing a film, wherein the film comprises a water-soluble polymer and an aromatic thermoplastic polyurethane, wherein the film has a degree of crystallinity greater than 25%; and bonding a portion of the film to a substrate to produce a laminate. In some embodiments, the method includes cutting or scoring a pattern in the laminate including a plurality of apertures. In some embodiments, the film is configured to expand when in contact with body fluid. In some embodiments, a region of the film adjacent the apertures are not bonded to the substrate.

Further aspects, objects, and advantages will become apparent upon consideration of the detailed description.

### BRIEF DESCRIPTION OF THE DRAWINGS

FIG. 1 is schematic illustration of a plurality of layers of a portion of an absorbent article, according to some embodiments of the present invention.

FIG. 2 is an illustration of a water-responsive film including a star-shaped aperture/valve pattern as it transitions between a contracted state and an expanded state, according to some embodiments of the present invention.

FIG. 3 is an illustration of a water-responsive film including a bulls-eye shaped aperture/valve pattern as it transitions between a contracted state and an expanded state, according to some embodiments of the present invention.

FIGS. 4A-4F are illustrations of example aperture/valve designs of a water-responsive film, according to some embodiments of the present invention.

FIG. 5 is a cross-sectional view of a portion of an absorbent article, according to some embodiments of the present invention.

FIG. 6 is a cross-sectional view of a portion of an absorbent article, according to some embodiments of the present invention.

FIG. 7A is a flow diagram of a method of producing a laminate according to some embodiments of the present invention.

FIG. 7B is an illustration of an exemplary laminate including a substrate bonded to a portion of the water-responsive film, according to some embodiments of the present invention.

FIGS. 8A and 8B are photographs of an exemplary absorbent article including a water-responsive film including a bulls-eye shaped pattern in a contracted state (FIG. 8A) and in an expanded state (FIG. 8B), according to some embodiments of the present invention.

### DETAILED DESCRIPTION

### Introduction

The present disclosure relates to absorbent articles including a water-responsive film (e.g., a topsheet that comprises the film) that permits rapid intake of fluid and limits flowback of the fluid. In addition to the water-responsive film, the absorbent article may include a backsheet, and an absorbent body interposed between the film and the backsheet. Each of the water-responsive film, backsheet, and absorbent body have a wearer-facing surface and a garment-facing surface. In some embodiments, a substrate is bonded to the wearer-facing surface of the water-responsive film to produce a laminate that forms the outermost surface of the absorbent article in contact with the user's skin. The water-responsive film may include one or more layers in a liquid deposition region of the absorbent article.

The water-responsive film described herein may include one or more apertures that can be utilized as a water-responsive one-way valve to allow fluid passage to other portions of the absorbent article (e.g., an absorbent body). The film is elastic and water sensitive such that the film expands when in contact with fluids (e.g., urine, menses, or other bodily fluid). When unexpanded, the film allows rapid passage of a fluid through the apertures of the film, and optionally into another layer of the absorbent article (e.g., an absorbent body of an absorbent article). As fluid flows through the apertures of the film, the film also absorbs some of the fluid, and the film expands. The expansion of the film closes the apertures that initially facilitated rapid passage of the fluid. The closed apertures block further fluid from flowing through the film as flowback. Upon drying of the film (i.e., desorption of the fluid), the film contracts, opening the apertures, and the film can allow passage of additional fluid.

The water-responsive film includes a water-soluble polymer and an elastomeric thermoplastic polyurethane (TPU). In particular, the water-responsive film includes a blend of two polymers: (1) a water-soluble polymer with a substantially crystalline morphology; and (2) an aromatic TPU. The dual attributes of elasticity and water-responsiveness are achieved by blending a synergistic combination of the water-soluble polymer and aromatic TPU to produce a film having a crystallinity of at least 25%. It was found that the combination of an aromatic TPU having a suitable strength and tensile modulus with a water-soluble polymer having a suitable crystallinity produces a film that expands after contact with a fluid, followed by contraction of the film towards its original dimensions upon drying or desorption.

Absorbent articles including the water-responsive film avoid the conventional correlation between rate of fluid intake and volume of flowback and provide rapid intake of fluid with limited flowback of the fluid. That benefit arises from the film's ability to expand when in contact with fluid. In the expanded state, the water-responsive film can block the flow of fluid through the film, including flowback of fluid. Due to the water-responsive expansion and contraction properties of the film, the mechanism to transition the valve from an open first state to a closed second state does not require any action or input other than the fluid itself, i.e., the valve automatically opens or closes to allow or prevent fluid passage. The state of the valve is determined by an amount of fluid in contact with the film. In this way, the valve can alternate between the first state (e.g., contracted dimensions, open apertures) and a second state (expanded dimensions, closed aperture) for rapid passage of fluid while preventing flowback.

In some embodiments, the water-responsive film may be bonded to a substrate (e.g., a non-woven) to form a laminate. The substrate can control expansion and contraction of the water-responsive film. The substrate can be bonded to the wearer-facing surface or the garment-facing surface of the water-responsive film. In some embodiments, the substrate is bonded to the wearer-facing surface of the water-responsive film in contact with the user's skin. The laminate may be scored or cut to include a pattern including one or more apertures. For example, the laminate may be laser-cut or stamped to provide one or more apertures having various geometries (e.g., circle, star-shaped, rectangular, square). In this way, the film of the laminate can be utilized as a water-responsive one-way valve that regulates fluid flow to the absorbent body. The apertures of the water-responsive film may be positioned in a liquid deposition region of the absorbent article. When the absorbent article is in contact with a fluid, expansion of the film material surrounding or adjacent to the apertures causes the apertures to close. After the fluid is dried or desorbed, the water-responsive film contracts to its original dimensions (or substantially the original dimensions), opening the apertures for additional fluid flow.

The water-responsive film facilitates good liquid retention to maintain a dry surface and thereby keeps the skin of the wearer dry. In some embodiments, the backsheet is liquid impermeable and prevents any fluids from the absorbent body from leaking through the absorbent article. In some embodiments, the individual elements may be bonded to each other to produce an absorbent article that has the desired comfort and performance level.

### Definitions

The terms “invention,” “the invention,” “this invention,” and “the present invention” used herein are intended to refer broadly to all of the subject matter of this patent application and the claims below. Statements containing these terms should be understood not to limit the subject matter described herein or to limit the meaning or scope of the patent claims below.

As used herein, the meaning of “a,” “an,” or “the” includes singular and plural references unless the context clearly dictates otherwise.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding of one element to another can occur via continuous or intermittent bonds.

As used herein, the term “liquid impermeable” refers to a layer or multi-layer laminate in which liquid body exudates, such as urine, will not pass through the layer or laminate, under ordinary use conditions.

As used herein, the term “liquid permeable” refers to any material that is not liquid impermeable.

As used herein, the term “superabsorbent” refers herein to a water-swellable, water-insoluble organic or inorganic material capable, under the most favorable conditions, of absorbing at least about 10 times its weight and, in some embodiments, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

As used herein, the term “thermoplastic” refers herein to a material which softens and which can be shaped when exposed to heat and which substantially returns to a non-softened condition when cooled.

As used herein, the “wearer-facing surface” refers to a surface of an article facing in the direction of the individual wearing the article.

As used herein, the “garment-facing surface” refers to a surface of an article facing in the direction of a garment worn with (i.e., usually over) the article.

As used herein, a “liquid deposition region” of an article refers to a region of the article where liquid is predominantly deposited when in typical use.

As used herein, the terms “machine direction” or “MD” generally refer to the direction in which a material is produced. The term “cross-machine direction” or “CD” refer to the direction perpendicular to the machine direction. Dimensions measured in the cross-machine direction are referred to as “width” dimension, while dimensions measured in the machine direction are referred to as “length” dimensions.

As used herein, the terms “elastomeric” and “elastic” and refer to a material that, upon application of a stretching force, is stretchable in at least one direction (such as the CD direction), and which upon release of the stretching force, contracts/returns to approximately its original dimension. For example, a stretched material may have a stretched length that is at least 50% greater than its relaxed unstretched length, and will recover to within at least 50% of its stretched length upon release of the stretching force. A hypothetical example would be a one (1) inch sample of a material that is stretchable to at least 1.50 inches and which, upon release of the stretching force, will recover to a length of at least 1.25 inches. Desirably, the material contracts or recovers at least 50%, and even more desirably, at least 80% of the stretched length.

As used herein the terms “extensible” or “extensibility” generally refer to a material that stretches or extends in the direction of an applied force by at least about 50% of its relaxed length or width. An extensible material does not necessarily have recovery properties. For example, an elastomeric material is an extensible material having recovery properties. A film may be extensible, but not have recovery properties, and thus, be an extensible, non-elastic material.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

### Absorbent Articles

The present disclosure relates to absorbent articles. For example, the absorbent articles can be personal care products. Personal care products of the present disclosure include, but are not limited to, feminine hygiene products like sanitary wipes and menses absorbing devices (e.g., sanitary napkins and tampons), infant and childcare products such as disposable diapers, absorbent underpants, and training pants, wound dressings such as bandages, incontinence products, products for wiping and absorbing oils, and the like. Suitable absorbent articles are described in more detail in U.S. Pat. No. 7,632,258.

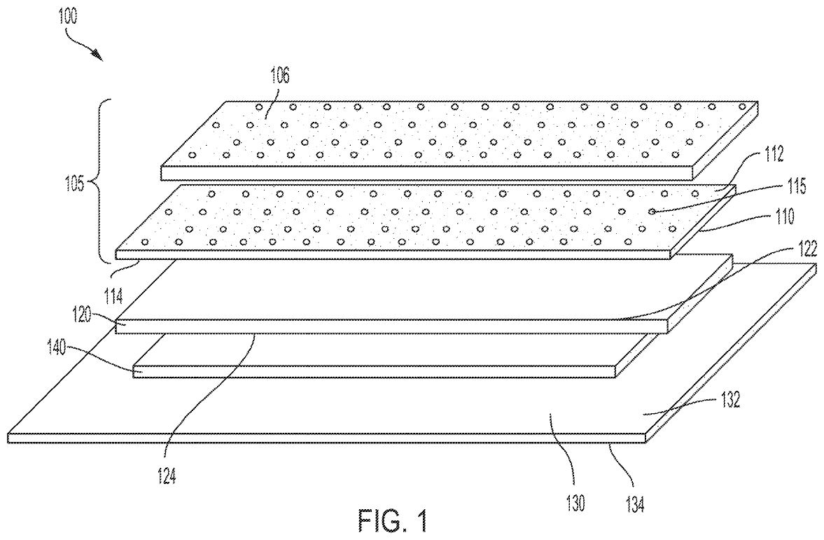

FIG. 1 is a schematic illustration of a portion of an absorbent article according to some embodiments of the present invention. The absorbent article 100 includes a plurality of layers that can be bonded to another layer (e.g., an adjacent layer or a non-adjacent layer). The absorbent article 100 illustrated in FIG. 1 includes a laminate 105 including a water-responsive film 110, a backsheet 130, and an absorbent body 120 positioned between the film 110 and the backsheet 130. Each of the film 110, absorbent body 120, and backsheet 130 includes a wearer-facing side and a garment-facing side. For example, the top surface of the film 110 is the wearer-facing surface 112 and the bottom surface of the film 110 is the garment-facing surface 114. As shown in FIG. 1, the wearer-facing surface is the top surface of the film 110, the absorbent body 120, and the backsheet 130 that is disposed toward or placed adjacent to the body of the wearer during ordinary use. The garment-facing surface is the bottom surface of the film 110, the absorbent body 120, and the backsheet 130 that is disposed away from the wearer's body and that faces in the direction of a garment worn over the absorbent article during ordinary use.

In FIG. 1, the garment-facing surface 114 of the film 110 may be directly adjacent to the wearer-facing side 122 of the absorbent body 120. The garment-facing side 124 of the absorbent body 120 may be directly adjacent or near to the wearer-facing side 132 of the backsheet 130. In some embodiments, the wearer-facing surface 112 of the film 110 is the outermost surface of the absorbent article 100 in direct contact with the user. In some embodiments, the garment-facing surface 134 of the backsheet 130 is the outermost surface of the absorbent article 100 and can directly contact a garment worn over the absorbent article. The film 110 allows fluids to flow to the absorbent body 120, and the backsheet 130 can be substantially impermeable to the fluids.

In some embodiments, the absorbent article 100 can also include one or more additional layers 140 such as that shown in FIG. 1. The additional layer 140 can be a liquid intake layer, a surge layer, a liquid wicking layer, a liquid distribution layer, a transfer layer, a barrier layer, and the like, as well as multiples and combinations thereof. In some embodiments, the additional layer 140 is disposed between the absorbent body 120 and the backsheet 130. The additional layer 140 can be provided in any position between each of the film 110, absorbent body 120, and backsheet 130. For example, the absorbent article 100 may include a fluid transfer layer between the laminate 105 and the absorbent body 120. Additionally, or alternatively, the absorbent article may include one or more additional layers (not shown) between the wearer-facing surface 112 of the film 110 and the outermost surface of the absorbent article 100.

In the embodiment of FIG. 1, the laminate 105 is a topsheet or a body-side liner of the absorbent article 100. The laminate 105 includes a substrate 106 bonded to the film 110. The film 110 is bonded to the substrate 106 to control the expansion direction of the film. In some embodiments, the wearer-facing surface of the film 110 is bonded to the substrate 106 to form the laminate 105. For example, the laminate 105 may comprise a wearer-facing surface 112 of the film 110 that is bonded to a non-woven as the substrate 106. In some embodiments, the non-woven can be in direct contact with the user's skin. In other embodiments, the laminate 105 could stand alone deeper in the absorbent article 100. In some embodiments, the film 110 could stand alone deeper in the absorbent article and can be bonded to another layer (e.g., the absorbent body). In some embodiments, the film 110 can be provided in any layer of the absorbent article 100 facing the wearer relative to the absorbent body 120.

The laminate 105 may be scored or cut to include a pattern. For example, the laminate 105 may be laser-cut or stamped to provide one or more apertures 150 having various geometries (e.g., circle, star-shaped, rectangular, square). In this way, the laminate 105 can be utilized as a water-responsive one-way valve for fluid flow to the absorbent body 120. The apertures 150 of the laminate 105 may be positioned in a liquid deposition region (not shown) of the absorbent article 100. Due to the expansion properties of the film 110, the material of the film 110 surrounding or adjacent to the plurality of apertures 150 may begin to expand and close the apertures 150 when the absorbent article 100 is in contact with a fluid. The film 110 enables the absorbent article to regulate fluid flow to the absorbent body 120. After the fluid is absorbed by the absorbent body 120 and desorbed from the film 110 (so the film is dry), the film 110 can contract to its original dimensions (or substantially the original dimensions) to open the apertures 150 for additional fluid flow.

In some embodiments, the film 110 is bonded to at least one substrate 106 to produce the laminate 105. For example, the film 110 may be adhesively bonded to at least one substrate 106. The film 110 can be bound to at least one substrate 106 (top and/or bottom) to control lateral expansion in the areas immediately surrounding the apertures 150, and thereby promote closing of the apertures 150 that function as a valve. This can be achieved by patterning the laminate 105 to leave a specific unbound area adjacent to the one or more apertures 150 of the film 110. The lateral expansion of the unbound portion of the film 110 allows the apertures 150 to close when contacted by water. The amount of expansion (e.g., lateral expansion) may be influenced by the composition of the film 110, the ratio of water-soluble polymer to thermoplastic polyurethane, and the amount of fluid contacting the laminate 105.

In some embodiments, a portion of the film 110 of the present disclosure can be treated with a hydrophobic agent or superhydrophobic agent that helps to keep fluids from sitting atop the wearer-facing surface 112 and leaving an unpleasant and/or unclean feeling from stains, accumulated debris, or wetness on the surface. For example, a region of the film 110 that is not adjacent the apertures 150 can be treated with a hydrophobic agent. The portions of film 110 treated with the hydrophobic agent can funnel the fluid to the apertures 150 of film 110. The apertures 150 ensure fluid permeability through open apertures when the film 110 is treated with hydrophobic or superhydrophobic agents. In some embodiments, the hydrophobic, superhydrophobic, or hydrophilic agents may be applied to a substrate bonded to the film 110 (e.g., a laminate or a liner).

In some embodiments, the area of the film 110 adjacent the apertures 150 can be treated with a hydrophilic treatment agent. The treatment agent can increase the surface energy in and around the apertures 150 to preferentially channel fluid flow into the apertures 150 to provide advantages of the present disclosure in protecting against stains, re-wetting, particulate debris accumulation on the wearer-facing surface 112 and other unpleasantries. In some embodiments, the film 110 is bonded to a substantially hydrophobic nonwoven. For example, the film 110 can be bonded to a spunbond, spunbond-meltblown-spunbond (SMS), Bonded Carded Web (BCW), spunlace, or coform.

In some embodiments, the surface of the film 110 can include a three-dimensional profile. For example, the film 110 may include 3-D materials with optimized apertures and combinations of hydrophilic and hydrophobic treatments strategically applied to the materials to improve absorption and transfer. 3-D structures can include bumps, pockets, raised areas, depressed areas, corrugations, and channels. Apertures 150 are typically disposed in the depressed areas or valleys between the bumps/corrugations/channels. In other aspects, the apertures 150 can be distributed uniformly or non-uniformly regardless of bump or valley. In general, the hydrophilic treatment agent is patterned such that it is disposed in the valleys, and the hydrophobic treatment agent is patterned such that it is disposed on the bumps or the raised portions of the topographical surface.

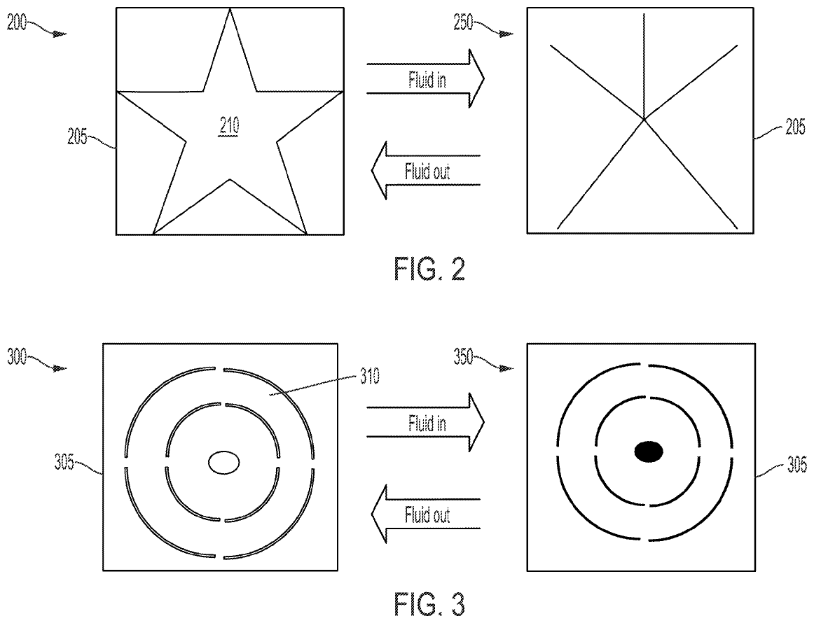

FIG. 2 shows an embodiment of a film described herein including an aperture. FIG. 2 illustrates the film as it transitions between a contracted state 200 and an expanded state 250. The film 205 includes one aperture 210, but the film 205 can include a plurality of apertures. As shown in the contracted state 200, the film 205 includes an aperture 210 that is star shaped. It is contemplated that the film 205 and the aperture 210 can have a plurality of different shapes to provide a desired flow rate or flow pattern through the apertures. For example, the apertures can be circular, ellipsoidal, rectangular, hexagonal, trapezoidal, T-shape, L-shape, hourglass shape, etc. In some embodiments, the film 205 can include multiple apertures that collectively form a pattern. The pattern of the apertures can provide a specific flow path for the fluid entering the absorbent article. For example, the pattern can include concentric circles in the liquid deposition region of the absorbent article. In some embodiments, the apertures comprise at least 10% of the total surface area of the film, e.g., at least 15%, at least 20%, at least 25%, at least 30%, at least 35%, at least 40%, at least 45%, at least 50%, or at least 55%. Liners that include apertures that comprise a larger surface can provide faster fluid intake.

In the contracted state 200, the film 205 is dry and the aperture 210 is open, providing an opening for fluid intake. As discussed herein, the crystalline regions of the water-soluble polymer are entangled with the aromatic thermoplastic polyurethane and pull the elastic aromatic thermoplastic polyurethane to a contracted state.

FIG. 2 also illustrates the film 205 in an expanded state 250 after it has absorbed water. In the expanded state 250, the expansion of the film 205 closes the aperture 210, which prevents fluid flowback. When the film 205 absorbs fluids (e.g., water), the water-soluble polymer begins to transition from a crystalline state to an amorphous state. The change in morphology of the water-soluble polymer from the contracted state 200 to the expanded state 250 effectively releases the elastic energy of the aromatic thermoplastic polyurethane, causing the film 205 to swell. In other words, the film 205 expands as the water-soluble polymer swells because of the entanglements of the thermoplastic polyurethane chains with the hydrophilic water-soluble polymer chains. Thus, the film 205 absorbs water from bodily fluid and the morphology of the water-soluble polymer transitions to the amorphous state for film expansion.

FIG. 3 shows another embodiment of the film including a bulls-eye shaped pattern for fluid flow through the film. In this embodiment, the film 305 is cut or scored to include a bulls-eye shaped pattern including a plurality of apertures 310 for fluid flow. FIG. 3 illustrates the transition of the film 305 from an open valve position in a contracted state 300 to a closed valve position in an expanded state 350.

The film 305 expands from the contracted state 300 to the expanded state 350 when it absorbs fluids (e.g., bodily fluid). When the film 305 dries due to desorption of the fluid, the film 305 retracts toward its original dimensions in the contracted state 300. During an insult, fluid will flow readily through the aperture 310. As a portion of the film 305 absorbs fluid, the film 305 begins to expand laterally along the edges of the aperture 310 and begins to close these areas. When the insult is completed, the film 305 continues to grow until it closes the aperture 310 as shown in the expanded state 350 of FIG. 3. By closing the aperture 310, the film 305 blocks passage of fluid through the valve in the opposite direction. After some time, the film 305 dries or desorbs fluid, and the film contracts toward its original dimensions in the contracted state 300, opening the valve for a possible repeat insult of fluid.

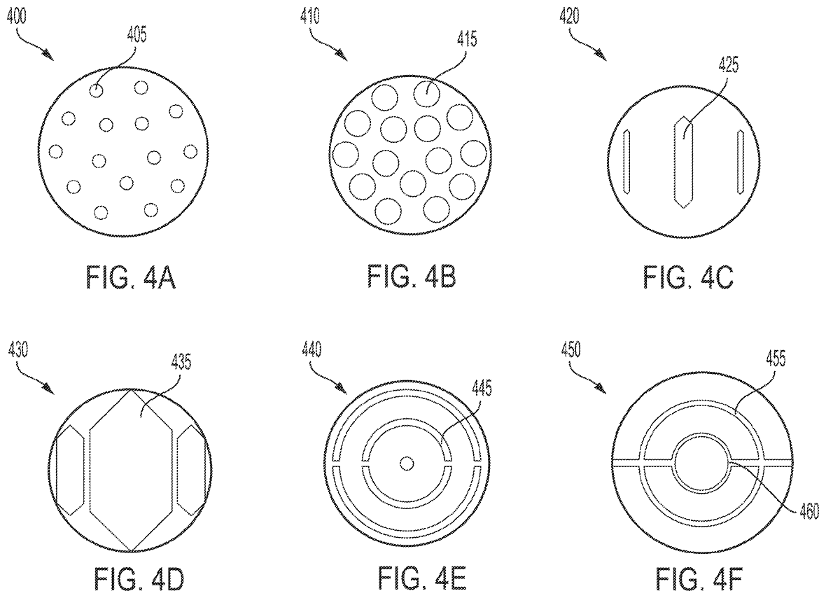

FIGS. 4A-4F provide multiple valve designs of the film according to some embodiments of the present invention. In some embodiments, the film may include an aperture having various geometries to control the fluid flow rate, the amount of absorption, and the fluid flowback. The total open area of the apertures in the films can be tailored for adequate fluid intake and valve closing based on the type of insult and insult location. In some embodiments, the film can be cut, stamped, or scored to form the apertures. For example, the film can be cut to include one or more slits. The one or more slits can form an intersecting pattern. In some embodiments, the intersecting pattern of the slits forms an aperture. Alternatively, the film can be produced with one or more apertures. In some embodiments, the film can be laser cut to provide a valve design in the film. A portion of the film can then be adhesively bonded to a substrate to control opening and closing of the valve. In some embodiments, the region adjacent the valve design (e.g., the region adjacent the one or more apertures) is not bonded to the substrate to allow for expansion of the film.

FIG. 4A shows a film including a plurality of apertures. In this embodiment, the film 400 includes a plurality of apertures 405 spaced apart on the film 400. The apertures 405 are substantially circular; however, the apertures can have any suitable geometry (e.g., square, ellipsoid, polygonal, etc.). The apertures 405 can be equidistantly spaced on the film 400. The size of the apertures 405 can be related to the flow rate of the fluid through the film. For example, FIG. 4B shows a film 410 including a plurality of circular apertures 415 that have a larger cross-sectional area than the apertures 405 of film 400. The film 410 is expected to have a higher flow rate of fluid than film 400.

FIG. 4C and FIG. 4D show films including a plurality of apertures having a hexagonal shape. The shape of the apertures 425 and 435 of films 420 and 430, respectively, are similar in length and width of a conventional pad. The apertures 425 of the film 420 have the same elongated hexagonal shape as the apertures 435 of the film 430. The apertures 425 of the film 420 of FIG. 4C have a smaller cross-sectional area in comparison to the apertures 435 of the film 430 of FIG. 4D. In this way, each of the films 420 and 430 can be tailored for instances where lower and higher flow rates are expected. FIGS. 4E and 4F show films including patterned apertures. The film 440 of FIG. 4E includes a bulls-eye shaped aperture 445. The film 450 of FIG. 4E includes a pattern comprising concentric circles 455 around a central aperture 460.

### Construction of Absorbent Article

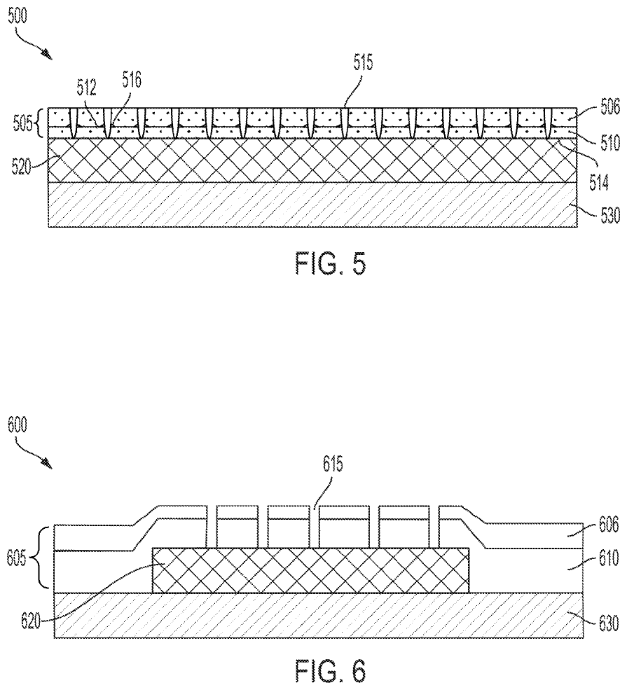

FIG. 5 provides a cross-sectional view of a portion of an absorbent article, according to some embodiments of the present invention. The absorbent article 500 includes a laminate 505, an absorbent body 520, and a backsheet 530. The laminate 505 includes a substrate 506 and a film 510. In some embodiments, the substrate 506 is a non-woven. The substrate 506 can be part of the topsheet or body-side liner of the absorbent article 500. The film 510 includes a wearer-facing surface 512 and a garment-facing surface 514. In some embodiments, the wearer-facing surface 512 of the film 510 is bonded to the substrate 506. In various embodiments, the laminate 505 of the absorbent article overlays an absorbent body 520 and a backsheet 530 and can isolate the wearer's skin from liquid waste retained by the absorbent body 520. In some embodiments, a fluid transfer layer can be positioned between the laminate 505 and the absorbent body 520. In some embodiments, the laminate 505 can be bonded to the optional fluid transfer layer, the absorbent body 520, and/or the backsheet 530. For example, the garment-facing surface 514 of the film 510 can be bonded to the fluid transfer layer via adhesive and/or by point fusion bonding. The point fusion bonding may be selected from ultrasonic, thermal, pressure bonding, and combinations thereof.

The laminate 505 can include a plurality of apertures 515. The apertures 515 extend through the substrate 506 and the film 510 of the laminate 505. In this way, the laminate 505 can control fluid flow to the absorbent body 520. In some embodiments, the apertures 515 can taper in width from the substrate 506 through the film 510. In some embodiments, the apertures 515 can have a uniform width through the laminate 505. A portion of the film 510 adjacent the aperture 515 may not be bonded to the substrate 506. For example, a region 516 adjacent the aperture at the interface of the substrate 506 and the film 510 is not adhesively bonded. This region 516 of the film 510 is not bonded to the substrate 506 to allow for expansion of the film 510 when in contact with bodily fluid. In some embodiments, the entire region 516 adjacent the aperture 515 of the film 510 is not bonded to the substrate 506. For example, in embodiments where the aperture is circular, the region of the film surrounding the circumference of the aperture is not bonded to the substrate.

FIG. 6 provides another cross-sectional view of a portion of an absorbent article, according to some embodiments of the present invention. FIG. 6 shows the film 610 of the laminate 605 substantially enclosing the absorbent body 620 between the backsheet 630 and the laminate 605. In some embodiments, the laminate 605 can extend beyond the absorbent body 620 and/or a fluid transfer layer (not shown) to overlay a portion of the backsheet 630 and can be bonded thereto by any method deemed suitable, such as, for example, by being bonded thereto by adhesive, to substantially enclose the absorbent body between the backsheet and the laminate. It is also contemplated that the laminate 605 may not extend beyond the absorbent body and/or may not be secured to the backsheet. It is further contemplated that the film 610 may be composed of more than one segment of material. The film 610 can be of different shapes, including rectangular, hourglass, or any other shape. The laminate 605 can be suitably compliant, soft feeling, and non-irritating to the wearer's skin and the film 610 can be the same as or less hydrophilic than the absorbent body 620 to permit body exudates to readily penetrate through to the absorbent body and provide a relatively dry surface to the wearer. The laminate 605 can include a plurality of apertures 615. The apertures 615 extend through the substrate 606 and the film 610 of the laminate 605 to the absorbent body 620.

### Film

The water-responsive film includes a water-soluble polymer and an aromatic thermoplastic polyurethane. The specific combination of the polymers, the morphology of the polymers, and the amounts of the polymers in the water-responsive film harness the elastic energy of the polymers to expand when in contact with water from bodily fluids. For example, the polymers can produce a water-responsive film having sufficient crystallinity such that the film can expand when in contact with a fluid and subsequently retract to its original state when dry. In this respect, the aromatic thermoplastic polyurethane is utilized as an elastomeric material that entangles the water-soluble polymer to provide for expansion of the film. The water-soluble polymer contributes to the crystallinity of the film such that when the film dries, the water-soluble polymer recrystallizes and retracts the film to its original dimensions. It was surprisingly and unexpectedly found that the combination of polymers described herein influences the ability and extent of water-responsive expansion and contraction.

For the film to have elastomeric properties for expansion and recovery, the polymers are tailored to provide a combination of elasticity and rigidity. For example, water-soluble polymer and an aromatic thermoplastic polyurethane are specifically tailored to produce a film having a crystallinity of at least 25%, e.g., at least 30%, at least 35%, at least 40%, at least 45%, at least 50%, or at least 55%. In some embodiments, the film may have a crystallinity from 25% to 60%, e.g., from 30% to 60%, from 30% to 55%, from 35% to 55%, from 35% to 50%, from 40% to 60%, from 45% to 60%, from 40% to 50%, or anywhere between these ranges. The water-soluble polymer mainly contributes to the crystalline nature of the film.

In some embodiments, the water-responsive film expands as it stays in contact with fluid until eventually the growth of the film reaches a maximum. After this time, continued contact with fluid results in the film beginning to contract. Without being bound by theory, it is believed that once a certain amount of the water-soluble polymer dissolves, decoupling of chain entanglements occurs, and the expanded film shrinks from dissolution. When the film is no longer in contact with the fluid, the remaining water-soluble polymer recrystallizes with the thermoplastic polyurethane chains, and the film retracts toward its original dimensions. The crystalline nature of the water-soluble polymer harnesses the elastic energy of the elastomer once again, and the film may expand if exposed to fluid a second time.

In some embodiments, the film may be mono- or multi-layered. Multilayer films may be prepared by co-extrusion of the layers, extrusion coating, or by any conventional layering process. In some embodiments, the multilayer films may include at least one base layer and at least one skin layer. In some embodiments, the multilayer films may include a plurality of layers. For example, the multilayer film may be formed from a base layer and one or more skin layers, wherein the base layer is formed from a blend of the water-soluble polymer and thermoplastic polyurethane. In some embodiments, the skin layer(s) are also formed from the polymer blend as described above. It should be understood, however, that other polymers may also be employed in the skin layer(s).

In some embodiments, the film is bonded to at least one substrate to produce a laminate. The film can be attached or bonded to at least one substrate. For example, the film may be adhesively bonded to at least one substrate. The film is bound to at least one substrate (top and/or bottom) to control lateral expansion and closing of the apertures that function as a valve. The laminate can include one or more apertures such that the film can function as a valve due to its expansion and contraction properties. For example, a portion of the substrate can be bonded to the film. This can be achieved by patterning (e.g., cutting or stamping) the laminate to leave a specific unbound area adjacent to the one or more apertures of the film bonded to the substrate. The lateral expansion of the unbound film allows the apertures to close when contacted by water. The amount of expansion (e.g., lateral expansion) may be influenced by the composition of the film, the ratio of water-soluble polymer to thermoplastic polyurethane, and the amount of fluid contacting the film.

For example, the film described herein can be bonded or attached to at least one substrate layer to regulate the amount of fluid flowing to the absorbent body. The film may include one or more apertures that allows for fluid intake to the absorbent structure. Fluid that contacts the film can pass through the apertures to the absorbent structure. A portion of the fluid can be absorbed by the film. The fluid absorbed by the film causes the film to expand and close the apertures, thereby serving as a valve to the absorbent structure.

### Thermoplastic Polyurethane

The film may include one or more aromatic TPUs. The aromatic TPU serves as an elastomeric material to allow the film to expand. The combination of the aromatic TPU and a substantially crystalline water-soluble polymer in the film leads to the surprising and unexpected result of film expansion after contact with bodily fluid, followed by contraction of the film towards its original dimensions upon drying or desorption. The ability of the film to expand when contacted by water and to shrink toward its original dimensions enables the film to close and open a valve (e.g., an aperture) responsively compared to films of the prior art, which only have the capability to shrink when contacted by water.

In some embodiments, the aromatic TPU polymer is hydrophilic. The TPU polymer may be present in the film in an amount from 10 wt. % to 90 wt. %, based on the total weight of the film, e.g., from 15 wt. % to 85 wt. %, from 20 wt. % to 80 wt. %, from 25 wt. % to 75 wt. %, from 30 wt. % to 70 wt. %, from 30 wt. % to 65 wt. %, from 30 wt. % to 70 wt. %, from 35 wt. % to 65 wt. %, or from 40 wt. % to 60 wt. %. In some embodiments, the film includes at least 10 wt. % TPU polymer, e.g., at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, or at least 70 wt. %. In some embodiments, the film includes from 40 wt. % to 60 wt. % TPU.

The film includes the aforementioned amounts of TPU in order to ensure that the elastic properties of the film allow for expansion. It was found that that the chemistry of the TPU contributes to the expansion properties of the film when in contact with water. Without being bound by theory, it is believed that when the TPU chains are entangled with a water-soluble polymer (e.g., polyethylene oxide), the hydrophilic aromatic TPU chains interact more extensively (through chain entanglements) with water-soluble polymer compared to the hydrophobic aliphatic TPU chains. As a result, when in contact with water, the crystalline regions of the water-soluble polymer begin to transition to a more amorphous state, which causes the polymer to swell. The change in morphology of the water-soluble polymer during swelling effectively releases the elastic energy of the aromatic TPU.

TPUs are generally synthesized from a polyol, organic diisocyanate, and optionally a chain extender. The synthesis of such melt-processable polyurethane elastomers may proceed either stepwise (e.g., prepolymer dispensing process) or by simultaneous reaction of all components in a single stage (e.g., one-shot dispensing process) as is known in the art and described in more detail in U.S. Pat. No. 3,963,656 to Meisert, et al.; U.S. Pat. No. 5,605,961 to Lee, et al.; U.S. Pat. No. 6,008,276 to Kalbe, et al.; U.S. Pat. No. 6,417,313 to Kirchmeyer, et al.; and U.S. Pat. No. 7,045,650 to Lawrey, et al., as well as U.S. Patent Application Publication Nos. 2006/0135728 to Peerlings, et al. and 2007/0049719 to Brauer, et al., all of which are incorporated by reference in their entireties for all intents and purposes

A polyol is generally any high molecular weight product having an active hydrogen component that may be reacted and includes materials having an average of about two or more hydroxyl groups per molecule. Long-chain polyols may be used that include higher polymeric polyols, such as polyester polyols and polyether polyols, as well as other acceptable “polyol” reactants, which have an active hydrogen component such as polyester polyols, polyhydroxy polyester amides, hydroxyl containing polycaprolactones, hydroxy-containing acrylic interpolymers, hydroxy-containing epoxies, and hydrophobic polyalkylene ether polyols. Typically, the polyol is substantially linear and has two to three hydroxyl groups, and a number average molecular weight of from 450 to 10,000, e.g., 450 to about 6000, or from 600 to 4500. Short-chain diols provide a harder, more crystalline polymer segment, and long-chain diols provide a softer, more amorphous polymer segment.

Suitable polyether diols may be produced by, for example, reacting one or more alkylene oxides having 2 to 4 carbon atoms in the alkylene residue with a starter molecule that contains two or more active hydrogen atoms in bound form. Exemplary alkylene oxides include ethylene oxide, 1,2-propylene oxide, epichlorohydrin and 1,2-butylene oxide and 2,3-butylene oxide. Exemplary starter molecules include water; aminoalcohols, such as N-alkyl-diethanolamines (e.g., N-methyl-diethanolamine); and diols, such as ethylene glycol, 1,3-propylene glycol, 1,4-butanediol and 1,6-hexanediol. In some embodiments, suitable polyester diols include ethanediol polyadipates, 1,4-butanediol polyadipates, ethanediol/1,4-butanediol polyadipates, 1,6-hexanedianeopentyl glycol polyadipates, 1,6-hexanediol/1,4-butanediol polyadipates and polycaproplactones.

The organic diisocyanates may include aromatic diisocyanates, such as 2,4- or 2,6-toluene diisocyanate, 4,4′-diphenylmethane diisocyanate, 2,4′-diphenylmethane diisocyanate, 2,2′-diphenylmethane diisocyanate, naphthylene-1,5-diisocyanate, xylylene diisocyanate, methylene diphenyl isocyanate (“MDI”), hexamethylene diisocyanate (“HMDI”), mixtures thereof, etc.

The chain extenders typically have a number average molecular weight of from about 60 to 400 and includes amino, thiol, carboxyl, and/or hydroxyl functional groups. In some embodiments, the chain extenders include two to three hydroxyl groups. As set forth above, one or more compounds selected from the aliphatic diols that contain from 2 to 14 carbon atoms may be used as the chain extender. Such compounds include, for example, ethanediol, 1,2-propanediol, 1,3-propanediol, 1,4-butanediol, 2,3-butanediol, 1,5-pentanediol, 1,6-hexanediol, diethylene glycol, dipropylene glycol, 1,4-cyclohexanediol, 1,4-dimethanolcyclohexane and neopentyl glycol. Diesters of terephthalic acid with glycols having 2 to 4 carbon atoms may also be employed. Some examples of such compounds include terephthalic acid bis-ethylene glycol and terephthalic acid bis-1,4-butanediol, hydroxyalkylene ethers of hydroquinone (e.g., 1-4-di(β-hydroxyethyl) hydroquinone), ethoxylated bisphenols (e.g., 1,4-di(β-hydroxyethyl)bisphenol A), (cyclo) aliphatic diamines (e.g., isophoronediamine, ethylendiamine, 1,2-propylenediamine, 1,3-propylenediamine, N-methyl-1,3-propylenediamine, and N,N′-dimethylethylene-diamine), and aromatic diamines (e.g., 2,4-toluenediamine, 2,6-toluenediamine, 3,5-diethyl-2,4-toluenediamine and 3,5-diethyl-2,6-toluenediamine, and primary mono-, di-, tri- or tetraalkyl-substituted 4,4′-diaminodiphenylmethanes).

In addition to those noted above, other components may also be employed to form the TPU. Catalysts, for instance, may be employed to facilitate formation of the polyurethane.

Suitable catalysts include, for instance, tertiary amines, such as triethylamine, dimethylcyclohexyl-amine, N-methylmorpholine, N,N′-dimethylpiperazine, 2-(dimethylaminoethoxy)-ethanol, diazabicyclo[2.2.2]octane, etc. as well as metal compounds, such as titanic acid esters, tin diacetate, tin dioctoate, tin dilaurate or the dialkyltin salts of aliphatic carboxylic acids such as dibutyltin diacetate or dibutyltin dilaurate or other similar compounds. Still other suitable additives that may be employed include light stabilizers (e.g., hindered amines), chain terminators, slip agents and mold release agents (e.g., fatty acid esters, the metal soaps thereof, fatty acid amides, fatty acid ester amides and silicone compounds), plasticizers, antiblocking agents, inhibitors, stabilizers against hydrolysis, heat and discoloration, dyes, pigments, inorganic and/or organic fillers, fungistatically and bacteriostatically active substances, fillers, etc.

In some embodiments, the TPU has a melting point of from 75° C. to 250° C., for example, from 100° C. to 240° C. or from 120° C. to 220° C. The glass transition temperature (“T_{g}”) of the TPU may be relatively low, for example, from −150° C. to 0° C., e.g., from −100° C. to −10° C., and from −85° C. to −20° C. The melting temperature and glass transition temperature may be determined using differential scanning calorimetry (“DSC”) in accordance with ASTM D-3417.

Examples of aromatic TPUs used in the films described herein include those available under the designation ESTANE™, PEARLSTICK™, and PEARLBOND™ from The Lubrizol Co. and under the designation WANTHANE® from Wanhua Chemical Group Co., Ltd. For example, ESTANE™ 58238, ESTANE™ EZ-44-61 EXP and WANTHANE® WHT-F170 are aromatic polyester-based polyurethanes that can be used in the films described herein. For example, ESTANET MVT 75AT3 is an aromatic polyether-based polyurethanes that can be used in the films described herein.

Other physical properties of the TPU may influence the ability and extent of water-responsive expansion of the films described herein. Table 1 provides several grades of TPUs and their chemical and physical properties. The tensile strength and tensile modulus of the TPU are important parameters to support robust expansion of the film. For example, an aromatic TPU having poor tensile strength and low molecular weight may not be able to support robust expansion of the film. On the other hand, if the tensile modulus is too high, the aromatic TPU may be too stiff for expansion and the TPUs may not have sufficient elasticity for expansion of the film.

In some embodiments, the tensile modulus of the TPU can range from 0.5 MPa to 8 MPa at an elongation of 100% as measured according to ASTM D-412 (2022), e.g., from 0.75 MPa to 7 MPa, from 0.9 MPa to 6 MPa, from 1 MPa to 5.5 MPa, from 1 MPa to 5 MPa, from 1.5 MPa to 4.5 MPa, or from 2 MPa to 4 MPa. In some embodiments, the tensile modulus of the TPU can range from 1 MPa to 5 MPa at an elongation of 100% as measured according to ASTM D-412 (2022).

In some embodiments, the tensile strength of the TPU can range from 5 MPa to 100 MPa, e.g., from 10 MPa to 80 MPa, from 15 MPa to 60 MPa, from 20 MPa to 50 MPa, from 25 MPa to 50 MPa, from 30 MPa to 60 MPa, or from 30 MPa to 50 MPa. In some embodiments, the tensile strength of the TPU can range from 20 MPa to 50 MPa.

In some embodiments, the shore hardness value the TPU can range from 50 to 90 as measured by ASTM D-2240 (2022), from 55 MPa to 85 MPa, from 60 MPa to 80 MPa, from 60 MPa to 75 MPa, from 65 MPa to 80 MPa, or from 70 MPa to 80 MPa. In some embodiments, the shore hardness value the TPU can range from 60 to 80.

TPU-1 denotes an aromatic polyether-based thermoplastic polymer. The product is available under the name ESTANE™ MVT 75AT3 (Lubrizol). The properties of the thermoplastic may be found below in Table 1.

TPU-2 denotes an aromatic polyester-based thermoplastic polymer. The product is available under the name WANTHANE® WHT-F170 (Wanhua). The properties of the thermoplastic may be found below in Table 1.

TPU-3 denotes an aromatic polyester-based thermoplastic polymer. The product is available under the name ESTANE™ 58238 (Lubrizol). The properties of the thermoplastic may be found below in Table 1.

TPU-4 denotes an aromatic polyester-based thermoplastic polymer. The product is available under the name ESTANE™EZ-44-61 EXP (Pearlbond 405 EXP) (Lubrizol). The properties of the thermoplastic may be found below in Table 1.

TPU-5 denotes an aromatic polyester-based thermoplastic polymer. The product is available under the name PEARLSTICK® 5702 F3 (Lubrizol). The properties of the thermoplastic may be found below in Table 1.

TPU-6 denotes an aromatic polyester-based thermoplastic polymer. The product is available under the name PEARLSTICK® 302 EXP (Lubrizol). The properties of the thermoplastic may be found below in Table 1.

TPU-7 denotes an aromatic polyester-based thermoplastic polymer. The product is available under the name PEARLSTICK® 305 EXP (Lubrizol). The properties of the thermoplastic may be found below in Table 1.

TPU-8 denotes an aliphatic polyether-based thermoplastic polymer. As used herein, the product may be used as a comparative example for comparing Aromatic based thermoplastic polymers to aliphatic thermoplastic polymers. The product is available under the name ESTANE™ AG 8451 (Lubrizol). The properties of the thermoplastic may be found below in Table 1.

TPU-9 denotes an aliphatic polyether-based thermoplastic polymer. As used herein, the product may be used as a comparative example for comparing Aromatic based thermoplastic polymers to aliphatic thermoplastic polymers. The product is available under the name ELASTOLLAN® LL1275A10 (BASF). The properties of the thermoplastic may be found below in Table 1.

TPU-10 denotes an aliphatic polyester-based thermoplastic polymer. As used herein, the product may be used as a comparative example for comparing Aromatic based thermoplastic polymers to aliphatic thermoplastic polymers. The product is available under the name ESTANE™ CLC93A-V (Lubrizol). The properties of the thermoplastic may be found below in

TABLE 1
Table 1.

| | | | | Tensile stress | Tensile stress | | |
|---|---|---|---|---|---|---|---|
| | | Hardness | Tensile | @ 100% | @ 300% | Ultimate | |
| Trade | Type of | (Shore | Strength | elongation- | elongation- | elongation | Size |
| Name | Polymer | A) | (MPa) | psi (MPa) | psi (MPa) | (%) | change |
| TPU-1 | Aromatic | 75 | 5000 | 600 | 900 | 700 | Expand |
| | Polyether | | (34.5) | (4.1) | (6.2) | | |
| TPU-2 | Aromatic | 72 | 3626 | 363 | 870 | 600 | Expand |
| | Polyester | | (25) | (2.5) | (6) | | |
| TPU-3 | Aromatic | 75 | 7000 | 650 | 1800 | 680 | Expand |
| | Polyester | | (48.3) | (4.5) | (12.4) | | |
| TPU-4 | Aromatic | 65 | 2500 | 380 | 540 | 1100 | Expand |
| | Polyester | | (17.2) | (2.62) | (3.7) | | |
| TPU-5 | Aromatic | 65 | 5366 | 290 | 725 | 590 | Expand |
| | Polyester | | (37) | (2) | (5) | | |
| TPU-6 | Aromatic | 55 | 2031 | 189 | 261 | 900 | Expand |
| | polyester- | | (14) | (1.3) | (1.8) | | |
| TPU-7 | Aromatic | 64 | 682 | 348 | 580 | 450 | Expand |
| | polyester- | | (4.7) | (2.4) | (4) | | |
| TPU-8 | Aliphatic | N/A | 7000 | 500 | 2000 | 475 | Shrink |
| | Polyether | | (48.3) | (3.45) | (13.8) | | |
| TPU-9 | Aliphatic | 70 | 5076 | 363 | 870 | 550 | Shrink |
| | Polyether | | (35) | (2.5) | (6) | | |
| TPU-10 | Aliphatic | 93 | 9000 | 1000 | N/A | 410 | Shrink |
| | Polyester | | (62.1) | (6.9) | | | |

Table 1 highlights the criticality of the chemistry of the TPU, which is evident by comparing the aliphatic and aromatic character of the TPU in the third column. Each of the TPUs in Table 1 were blended with a water-soluble polymer and extruded into a film to investigate the effect of the TPU on the properties of the film. Each of the films produced from an aromatic TPU expanded when in contact with water, whereas films produced from aliphatic TPU, both polyether based and polyester based, shrank when in contact with water. TPU-9, an aliphatic polyether-based TPU, had similar physical properties to TPU-1, an aromatic polyether-based TPU, and TPU-2, an aromatic polyester-based TPU. However, the film including the aliphatic polyether TPU-9 shrank, while the films including the aromatic polyether TPU-1 and the aromatic polyester TPU-2 both expanded.

TPU-8, an aliphatic polyether-based TPU, had similar physical properties to TPU-3, an aromatic polyester-based TPU. Once again, the films produced from the aromatic polyether-based TPU expanded while the films including an aliphatic polyether-based TPU shrank. All the aromatic TPUs are examples of elastomers that may be used in the water-responsive expandable film described herein. It is contemplated that other aromatic TPUs with similar physical properties to those in Table 1 represent additional exemplary elastomeric components that can be used in the films described herein.

### Water-Soluble Polymer

The film also includes one or more water-soluble polymers. The water-soluble polymer may be present in the film in an amount from 10 wt. % to 90 wt. %, based on the total weight of the film, e.g., from 15 wt. % to 85 wt. %, from 20 wt. % to 80 wt. %, from 25 wt. % to 75 wt. %, from 30 wt. % to 70 wt. %, from 30 wt. % to 65 wt. %, from 30 wt. % to 70 wt. %, from 35 wt. % to 65 wt. %, or from 40 wt. % to 60 wt. %. In some embodiments, the film includes at least 10 wt. % of water-soluble polymer, e.g., at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, or at least 70 wt. %. In some embodiments, the film includes water-soluble polymer in an amount from 40 wt. % to 60 wt. %, based on the total weight of the film.

In some embodiments, the water-soluble polymer has a substantially crystalline (e.g., degree of crystallinity of at least 50%) morphology. The expansion and contraction properties of the film are partially dependent on the morphology and molecular weight of the water-soluble polymer. For example, the crystalline morphology of the water-soluble polymer enables the water-soluble polymer to transition from the crystalline state to the amorphous state when in contact with water. When the film is no longer in contact with the fluid, the remaining water-soluble polymer recrystallizes with the TPU chains, and the film retracts toward its original dimensions after desorption or drying. The crystalline nature of the water-soluble polymer “locks” the elastic energy of the elastomer (e.g., TPU).

The water-soluble polymer contributes to the crystallinity of the film. In some embodiments, the water-soluble polymer comprises a crystallinity greater than 50%. For example, the water-soluble polymer comprises a crystallinity greater than 50%, greater than 55%, greater than 60%, greater than 65%, greater than 70%, greater than 75%, greater than 80%, greater than 85%, or greater than 90%. In some embodiments, the water-soluble polymer comprises a crystallinity from 50% to 95%, e.g., from 55% to 95%, from 60% to 95%, from 65% to 90%, from 70% to 90%, from 75% to 90%, or from 80% to 95%.

In some embodiments, the water-soluble polymers employed in the films described herein generally have a high molecular weight. For example, the water-soluble polymers may have a weight average molecular weight (Mw) greater than 25,000, greater than 50,000, greater than 75,000, greater than 100,000, greater than 125,000, greater than 150,000, greater than 175,000, greater than 200,000, greater than 225000, or greater than 250,000. In some embodiments, the water-soluble polymers has a weight average molecular weight (Mw) ranging from 25,000 to 500,000 grams per mole, e.g., from 50,000 to 475,000 grams per mole, from 75,000 to 450,000 grams per mole, from 80,000 to 400,000 grams per mole, from 90,000 to 350,000 grams per mole, from 100,000 to 300,000 grams per mole, from 100,000 to 250,000 grams per mole, from 100,000 to 200,000 grams per mole, or from 150,000 to 250,000 grams per mole. In some embodiments, the water-soluble polymer is polyethylene oxide. In some embodiments, the polyethylene oxide has a weight average molecular weight (Mw) greater than 25,000, greater than 50,000, greater than 75,000, greater than 100,000, greater than 125,000, greater than 150,000, greater than 175,000, greater than 200,000, greater than 225000, or greater than 250,000.

As described herein, the water-soluble polymer and aromatic TPU are specifically tailored to produce a film having a crystallinity from 25% to 60%. The water-soluble polymer mainly contributes to the crystalline nature of the film. In some embodiments, the water-soluble polymer may comprise polyethylene oxide, hydroxypropyl cellulose, polyvinyl alcohol, polyvinyl pyrrolidone, polyvinyl pyridine, gelatinized starch, nylon copolymers, polyacrylic acid, and mixtures thereof.

PEO-1 denotes a water-dispersible polyethylene oxide polymer. The product is available under the name POLYOX™ WSR N-80 and available from DuPont, Inc.

PEO-2 denotes a water-dispersible polyethylene oxide polymer. The product is available under the name POLYOX™ WSR N-10 and available from DuPont, Inc.

PVOH-1 denotes a water-dispersible polyvinyl alcohol polymer. The product is available under the name NICHIGO G-POLYMER™ OKS-8035 and available from Soarus, Inc.

In some embodiments, the water-soluble polymer can be polyethylene oxide (PEO). For example, a commercially available polyethylene oxide with high crystallinity is PEO-1. Table 2 provides the thermal analysis of films including PEO-1 and TPU-2, which is an aromatic TPU. The film including 100 wt. % polyethylene oxide exhibited a crystallinity of 77% measured by differential scanning calorimetry (DSC) thermal analysis. Each of the compositions including a blend of PEO-1 and TPU-2 exhibited sufficient crystallinity to expand when in contact with water. The films including 100 wt. % TPU did not exhibit any crystallinity based on the DSC analysis due to their amorphous morphology.

TABLE 2

| | Melting Point °C | Xc (%) | T_{g} (°C) |
|---|---|---|---|
| 100% PEO | 65.6 | 77.0 | — |
| 80% PEO-1/20% | 63.0 | 61.0 | −44.5 |
| TPU-2 | | | |
| 70% PEO-1/30% | 61.9 | 56.0 | −45.5 |
| TPU-2 | | | |
| 60% PEO-1/40% | 60.9 | 47.8 | −46.3 |
| TPU-2 | | | |
| 100% TPU-2 | — | — | −45.7 |

In some embodiments, the water-soluble polymers may be formed from monomers such as vinyl pyrrolidone, hydroxyethyl acrylate or methacrylate (e.g., 2-hydroxyethyl methacrylate), hydroxypropyl acrylate or methacrylate, acrylic or methacrylic acid, acrylic or methacrylic esters or vinyl pyridine, acrylamide, vinyl acetate, vinyl alcohol (hydrolyzed from vinyl acetate), ethylene oxide, derivatives thereof, and so forth. Other examples of suitable monomers are described in U.S. Pat. No. 4,499,154 to James, et al., which is incorporated by reference in its entirety for all intents and purposes. The resulting polymers may be homopolymers or interpolymers (e.g., copolymer, terpolymer, etc.), and may be nonionic, anionic, cationic, or amphoteric. In addition, the polymer may be of one type (i.e., homogeneous), or mixtures of different polymers may be used (i.e., heterogeneous). In one particular embodiment, the water-soluble polymer contains a repeating unit having a functional hydroxyl group, such as polyvinyl alcohol (“PVOH”), copolymers of polyvinyl alcohol (e.g., ethylene vinyl alcohol copolymers, methyl methacrylate vinyl alcohol copolymers, etc.). Vinyl alcohol polymers, for instance, have at least two or more vinyl alcohol units in the molecule and may be a homopolymer of vinyl alcohol, or a copolymer containing other monomer units.

The degree of hydrolysis may be selected to optimize solubility, etc., of the water-soluble polymer. For example, the degree of hydrolysis may be from 60 mole % to 95 mole %, e.g., from 80 mole % to 90 mole % or from 85 mole % to 89 mole %. Examples of suitable partially hydrolyzed polyvinyl alcohol polymers are available under the designation CELVOL™ 203, 205, 502, 504, 508, 513, 518, 523, 530, or 540 from Celanese Corp. Other suitable partially hydrolyzed polyvinyl alcohol polymers are available under the designation ELVANOL™ 50-14, 50-26, 50-42, 51-03, 51-04, 51-05, 51-08, and 52-22 from DuPont.

The relative amount of the water-soluble polymer and TPU employed in the film may also be selected to help further optimize expansion and retraction properties of the film. In some embodiments, the weight ratio of the water-soluble polymer to the TPU is from 0.5:1 to 8:1, e.g., from 1:1 to 7:1, from 1.25:1 to 6:1, from 1.5:1 to 5:1, from 1.5:1 to 4:1, from 1.5:1 to 3:1, from 1.5:1 to 2.5:1, from 1:1 to 3:1, or from 1.25:1 to 2:1. The TPU may constitute from 10 wt. % to 90 wt. %, e.g., from 15 wt. % to 60 wt. % or from about 20 wt. % to about 50 wt. %, based on the total weight of the film. The water-soluble polymer may constitute from 10 wt. % to 90 wt. %, e.g., from 20 wt. % to 80 wt. % or from 40 wt. % to 70 wt. %, based on the total weight of the film.

### Additives

In some embodiments, the film may optionally include one or more additives. In some embodiments, the additives may include surfactants, absorbents, plasticizer, minerals, antibiotics, or skin benefit agents. For example, the film may include superabsorbent polymers. The superabsorbent polymers are generally employed to increase the absorbent capacity of the film and expansion. In some embodiments, the superabsorbent polymers are present in the film in the form of small particles. Superabsorbent polymeric powders suitable for the film include, but are not limited to, a wide variety of anionic, cationic, and nonionic materials. Suitable polymers include polyacrylamides, polyvinyl alcohols, ethylene maleic anhydride copolymer, polyvinylethers, polyacrylic acids, polyvinylpyrrolidones, polyvinylmorpholines, polyamines, polyethyleneimines, polyquaternary ammoniums, natural based polysaccharide polymers such as carboxymethyl celluloses, carboxymethyl starches, hydroxypropyl celluloses, algins, alginates, carrageenans, acrylic grafted starches, acrylic grafted celluloses, chitin, chitosan, and synthetic polypeptides such as polyaspartic acid, polyglutamic acid, polyasparagins, polyglutamines, polylysines, and polyarginines, as well as the salts, copolymers, and mixtures of any of the foregoing polymers.

In addition to the components noted above, other additives may also be incorporated into the liner, such as slip additives (e.g., fatty acid salts, fatty acid amides, etc.), compatibilizers (e.g., functionalized polyolefins), dispersion aids, melt stabilizers, processing stabilizers, heat stabilizers, light stabilizers, antioxidants, heat aging stabilizers, whitening agents, antiblocking agents, bonding agents, lubricants, fillers, etc.

In some embodiments, the film may include fillers. Fillers are particulates or other forms of material that may be added to the film polymer extrusion blend and that will not chemically interfere with the extruded film, but which may be uniformly dispersed throughout the film. Fillers may serve a variety of purposes, including enhancing film opacity and/or breathability (i.e., vapor-permeable and substantially liquid-impermeable). For instance, filled films may be made breathable by stretching, which causes the polymer to break away from the filler and create microporous passageways. Breathable microporous elastic films are described, for example, in U.S. Pat. Nos. 5,997,981; 6,015,764; and 6, 111, 163 to McCormack, et al.; U.S. Pat. No. 5,932,497 to Morman, et al.; U.S. Pat. No. 6,461,457 to Taylor, et al., all of which are incorporated by reference in their entireties for all intents and purposes. Further, hindered phenols are commonly used as an antioxidant in the production of films. Some suitable hindered phenols include those available from Ciba Specialty Chemicals under the trade name “Irganox®”, such as Irganox® 1076, 1010, or E 201. Moreover, bonding agents may also be added to the film to facilitate bonding of the film to additional materials (e.g., nonwoven webs). Examples of such bonding agents include hydrogenated hydrocarbon resins. Other suitable bonding agents are described in U.S. Pat. No. 4,789,699 to Kieffer et al. and U.S. Pat. No. 5,695,868 to McCormack, which are incorporated by reference in their entireties for all intents and purposes.

### Backsheet

In some embodiments, the absorbent article includes a backsheet. The backsheet and/or portions thereof can be liquid impermeable and optionally breathable. The backsheet and/or portions thereof can be elastic, stretchable, or non-stretchable. The backsheet may be constructed of a single layer, multiple layers, laminates, spunbond fabrics, films, liners, meltblown fabrics, elastic netting, microporous webs, bonded-carded webs or foams provided by elastomeric or polymeric materials. In some embodiments, the backsheet can be constructed of a microporous polymeric film, such as polyethylene or polypropylene. In some embodiments, the backsheet can be a single layer of a liquid impermeable material. In some embodiments, the backsheet can be suitably stretchable, and more suitably elastic, in at least the lateral or circumferential direction of the absorbent article.

In some embodiments, the backsheet can be a multi-layered laminate in which at least one of the layers is liquid impermeable. In some embodiments, the backsheet can be a two-layer construction, including an outer layer material and an inner layer material which can be bonded together such as by a laminate adhesive. Suitable laminate adhesives can be applied continuously or intermittently as beads, a spray, parallel swirls, or the like. Suitable adhesives can be obtained from Bostik Findlay Adhesives, Inc. of Wauwatosa, WI, U.S.A. It is to be understood that the inner layer can be bonded to the outer layer by other bonding methods, including, but not limited to, ultrasonic bonds, thermal bonds, pressure bonds, or the like.

The outer layer of the backsheet can be any suitable material and may be one that provides a generally cloth-like texture or appearance to the wearer. An example of such material can be a 100% polypropylene bonded-carded web with a diamond bond pattern available from Sandler A. G., Germany, such as 30 gsm Sawabond 4185® or equivalent. Another example of material suitable for use as an outer layer of a backsheet can be a 20 gsm spunbond polypropylene non-woven web. The liquid impermeable inner layer of the backsheet (or the liquid impermeable backsheet where the backsheet is of a single-layer construction) can be either vapor permeable (i.e., “breathable”) or vapor impermeable. The liquid impermeable inner layer (or the liquid impermeable backsheet where the backsheet is of a single-layer construction) may be manufactured from a thin plastic liner, although other liquid impermeable materials may also be used. The liquid impermeable inner layer (or the liquid impermeable backsheet where the backsheet is of a single-layer construction) can inhibit liquid body exudates from leaking out of the absorbent article and wetting articles, such as bed sheets and clothing, as well as the wearer and caregiver. An example of a material for a liquid impermeable inner layer (or the liquid impermeable backsheet where the backsheet is of a single-layer construction) can be a printed 19 gsm Berry Plastics XP-8695H liner or equivalent commercially available from Berry Plastics Corporation, Evansville, IN, U.S.A. Where the backsheet is of a single layer construction, it can be embossed and/or matte finished to provide a more cloth-like texture or appearance. The backsheet can permit vapors to escape from the absorbent article while preventing liquids from passing through. A suitable liquid impermeable, vapor permeable material can be composed of a microporous polymer liner or a non-woven material which has been coated or otherwise treated to impart a desired level of liquid impermeability.

### Absorbent Body

The absorbent body can be suitably constructed to be generally compressible, conformable, pliable, non-irritating to the wearer's skin and capable of absorbing and retaining fluids. The absorbent body can be manufactured in a wide variety of sizes and shapes (for example, rectangular, trapezoidal, T-shape, I-shape, hourglass shape, etc.) and from a wide variety of materials. The size and the absorbent capacity of the absorbent body should be compatible with the size of the intended wearer and the liquid loading imparted by the intended use of the absorbent article. Additionally, the size and the absorbent capacity of the absorbent body can be varied to accommodate wearers ranging from infants to adults.

The absorbent body can have garment-facing surface and wearer-facing surface. In some embodiments, the wearer-facing surface may be adjacent the film and the garment-facing surface may be adjacent the backsheet. In some embodiments, the absorbent body can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In some embodiments, the absorbent body can be a matrix of cellulosic fluff and superabsorbent material.

In some embodiments, the absorbent body may be constructed of a single layer of materials, or in the alternative, may be constructed of two or more layers of materials. In some embodiments in which the absorbent body has two layers. The absorbent body can have a wearer-facing layer and/or a garment-facing layer comprising a superabsorbent material. In some embodiments, the absorbent body can have a wearer-facing layer suitably composed of hydrophilic fibers and a garment-facing layer suitably composed at least in part of a high absorbency material commonly known as superabsorbent material. In such an embodiment, the wearer-facing layer of the absorbent body can be suitably composed of cellulosic fluff, such as wood pulp fluff, and the garment-facing layer of the absorbent body can be suitably composed of superabsorbent material, or a mixture of cellulosic fluff and superabsorbent material. As a result, the wearer-facing layer can have a lower absorbent capacity per unit weight than the garment-facing layer. The wearer-facing layer may alternatively be composed of a mixture of hydrophilic fibers and superabsorbent material. It is also contemplated that, in some embodiments, the garment-facing layer may be composed solely of superabsorbent material without departing from the scope of this disclosure.

Various types of wettable, hydrophilic fibers can be used in the absorbent body. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In some embodiments, the cellulosic fluff can include a blend of wood pulp fluff. The absorbent body can be formed with a dry-forming technique, an air-forming technique, a wet-forming technique, a foam-forming technique, or the like, as well as combinations thereof. A coform nonwoven material may also be employed. Methods and apparatus for carrying out such techniques are well known in the art. Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. Typically, a superabsorbent material can be capable of absorbing at least about ten times its weight in liquid. In some embodiments, the superabsorbent material can absorb up to forty times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes having a largest greatest dimension/smallest dimension ratio, such as needles, flakes, and fibers are also contemplated for use herein. Conglomerates of particles of superabsorbent materials may also be used in the absorbent body.

In some embodiments, the absorbent body can have at least about 15 wt. % of a superabsorbent material, based on the total weight of the absorbent body. In some embodiments, the absorbent body can have at least about 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 99 wt. % or 100 wt. % of a superabsorbent material. In some embodiments, the absorbent body can be free of superabsorbent material.

### Fluid Transfer Layer

In some embodiments, the absorbent article may include a fluid transfer layer. In some embodiments, the fluid transfer layer can be in contact with the absorbent body. In some embodiments, the fluid transfer layer can be bonded to the absorbent body. Bonding of the fluid transfer layer to the absorbent body can occur via any means known to one of ordinary skill, such as, but not limited to, adhesives. In some embodiments, a fluid transfer layer can be positioned between the film and the absorbent body. In some embodiments, a fluid transfer layer can completely encompass the absorbent body and can be sealed to itself. In such an embodiment, the fluid transfer layer may be folded over on itself and then sealed using, for example, heat and/or pressure. In some embodiments, a fluid transfer layer may be composed of separate sheets of material which can be utilized to partially or fully encompass the absorbent body and which can be sealed together using a sealing means such as, but not limited to, an ultrasonic bonder or other thermochemical bonding means or the use of an adhesive.

In some embodiments, the fluid transfer layer can be in contact with and/or bonded with the wearer-facing surface of the absorbent body. In some embodiments, the fluid transfer layer can be in contact with and/or bonded with the wearer-facing surface and at least one of the edges of the absorbent body. In some embodiments, the fluid transfer layer 72 can be in contact with and/or bonded with the wearer-facing surface, at least one of the edges of the absorbent body, and the garment-facing surface of the absorbent body. In some embodiments, the absorbent body may be partially or completely encompassed by a fluid transfer layer.

The fluid transfer layer can be pliable, less hydrophilic than the absorbent body, and sufficiently porous to thereby permit liquid body exudates to penetrate through the fluid transfer layer to reach the absorbent body. In some embodiments, the fluid transfer layer can have sufficient structural integrity to withstand wetting thereof and of the absorbent body. In some embodiments, the fluid transfer layer can be constructed from a single layer of material or it may be a laminate constructed from two or more layers of material.

In some embodiments, the fluid transfer layer can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp. Wood pulps can include, but are not limited to, standard softwood fluffing grade, which is a bleached, highly absorbent sulfate wood pulp containing primarily southern soft wood fibers. In some embodiments, the fluid transfer can include a through-air bonded carded web comprised of synthetic fibers. For example, the synthetic fibers may include monocomponent polyolefinic fibers or polyester fibers. In some embodiments, the fluid transfer can also include bicomponent fibers comprising polyolefins and/or polyester.

In various embodiments, the fluid transfer layer can include cellulosic material. In various embodiments, the fluid transfer layer can be creped wadding or a high-strength tissue. In various embodiments, the fluid transfer layer can include polymeric material. In some embodiments, a fluid transfer layer can include a spunbond material. n some embodiments, a fluid transfer layer can include a meltblown material. n some embodiments, the fluid transfer layer can be a laminate of a meltblown nonwoven material having fine fibers laminated to at least one spunbond nonwoven material layer having coarse fibers. In such an embodiment, the fluid transfer layer can be a spunbond-meltblown (“SM”) material. In some embodiments, the fluid transfer layer can be a spunbond-meltblown-spunbond (“SMS”) material. non-limiting example of such a fluid transfer layer can be a 10 gsm SMS material.

In some embodiments, the fluid transfer layer can be composed of at least one material which has been hydraulically entangled into a nonwoven substrate. In some embodiments, the fluid transfer layer can be composed of at least two materials which have been hydraulically entangled into a nonwoven substrate. In some embodiments, the fluid transfer layer can have at least three materials which have been hydraulically entangled into a nonwoven substrate. A non-limiting example of a fluid transfer layer can be a 33 gsm hydraulically entangled substrate. In such an example, the fluid transfer layer can be a 33 gsm hydraulically entangled substrate composed of a 12 gsm spunbond material, a 10 gsm wood pulp material having a length from about 0.6 cm to about 5.5 cm, and an 11 gsm polyester staple fiber material. To manufacture the fluid transfer layer just described, the 12 gsm spunbond material can provide a base layer while the 10 gsm wood pulp material and the 11 gsm polyester staple fiber material can be homogeneously mixed together and deposited onto the spunbond material and then hydraulically entangled with the spunbond material.

In some embodiments, a wet strength agent can be included in the fluid transfer layer. A non-limiting example of a wet strength agent can be Kymene 6500 (557LK) or equivalent available from Ashland Inc. of Ashland, KY, U.S.A. In some embodiments, a surfactant can be included in the fluid transfer layer. In some embodiments, the fluid transfer layer can be hydrophilic. In some embodiments, the fluid transfer layer can be hydrophobic and can be treated in any manner known in the art to be made hydrophilic.

In some embodiments, the fluid transfer layer can be in contact with and/or bonded with an absorbent body which is made at least partially of particulate material such as superabsorbent material. In some embodiments in which the fluid transfer layer at least partially or completely encompasses the absorbent body, the fluid transfer layer should not unduly expand or stretch as this might cause the particulate material to escape from the absorbent body. In some embodiments, the fluid transfer layer, while in a dry state, can have respective extension values at peak load in the machine and cross directions of 30 percent or less and 40 percent or less, respectively.

### Method of Producing a Liner

Any known technique may be used to form a film described herein, including blowing, casting, flat die extruding, etc. In some embodiments, the film may be formed by a blown process in which a gas (e.g., air) is used to expand a bubble of the extruded polymer blend through an annular die. The bubble is then collapsed and collected in flat film form. Processes for producing blown films are described, for instance, in U.S. Pat. No. 3,354,506 to Raley; U.S. Pat. No. 3,650,649 to Schippers; and U.S. Pat. No. 3,801,429 to Schrenk et al., as well as U.S. Patent Application Publication Nos. 2005/0245162 to McCormack, et al. and 2003/0068951 to Boggs, et al., all of which are incorporated by reference in their entireties for all intents and purposes. In another embodiment, the film is formed using a casting technique.

In some embodiments, the film can be formed by blending each of the components together using any of a variety of known techniques. In one embodiment, for example, the components may be supplied separately or in combination. For instance, the components may first be dry mixed together to form an essentially homogeneous mixture, and they may likewise be supplied either simultaneously or in sequence to a melt processing device (e.g., extruder) that uniformly blends the materials.

In some embodiments, the methods for producing the film may include batch and/or continuous melt processing techniques. For example, a mixer/kneader, Banbury mixer, Farrel continuous mixer, single-screw extruder, twin-screw extruder, roll mill, etc., may be utilized to blend and melt process the materials. Examples of suitable melt processing devices may include a co-rotating, twin-screw extruder (e.g., USALAB twin-screw extruder available from Thermo Electron Corporation of Stone, England or an extruder available from Werner-Pfreiderer from Ramsey, N.J.). Such extruders may include feeding and venting ports and provide high intensity distributive and dispersive mixing. For example, the components may be fed to the same or different feeding ports of the twin-screw extruder and melt blended to form a substantially homogeneous melted mixture. If desired, other additives may also be injected into the polymer melt and/or separately fed into the extruder at a different point along its length.

In some embodiments, the film can be produced in a melt-blending device. For example, the water-soluble polymer and TPU can be melt-blended and extruded in an extruder. The water-soluble polymer and TPU can be separately supplied to an extruder where they are uniformly blended. In some embodiments, the film can be produced using a single-screw extruder or a twin-screw extruder (e.g., co-rotating, twin-screw extruder). Commercially available single-screw extruders suitable for producing the film include, for example, HAAKE™ Rheomex OS Single Screw Extruder from Thermo Fisher Scientific, Waltham, MA. Commercially available twin-screw extruder include, for example, a ZSK-30 extruder available from Werner & Pfleiderer Corporation of Ramsey, N.J., or a Thermo Prism™ USALAB 16 extruder available from Thermo Electron Corp., Stone, England. In some embodiments, melt blending may occur at a temperature of from 50° C. to 300° C., e.g., from 75° C. to 250° C., from 100° C. to 225° C., from 120° C. to 215° C. or from 120° C. to 200° C. In some embodiments, the screw speed of the extruder may be up to 200 RPM. The polymer melt from the extruder can be processed in the film die to produce the film. In some embodiments, the film can be collected on one or more chill rollers.

In some embodiments, the film can be produced in a two-step process including compounding and extruding the film. In this embodiment, the water-soluble polymer and TPU can be compounded into pellets, and the pellets can be extruded to produce the film. For example, the water-soluble polymer and TPU can be melt blended via the twin screw extruder at an extrusion temperature to form a homogeneous polymer blend. The molten polymer blend can then be extruded through a filament die to produce a sheet. Thereafter, the extruded material may be chilled and cut into pellet form. In some embodiments, the extruded material can be air-cooled on a conveyor using fans, and then cut into pellets using a pelletizing system. The compounded pellets can be melted in another twin-screw extruder at an extrusion temperature and extruded through a film die onto chill rollers.

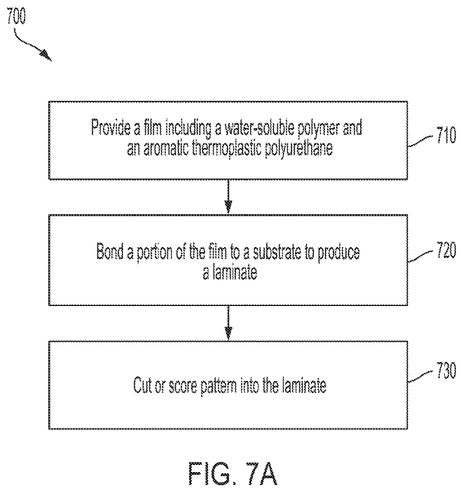

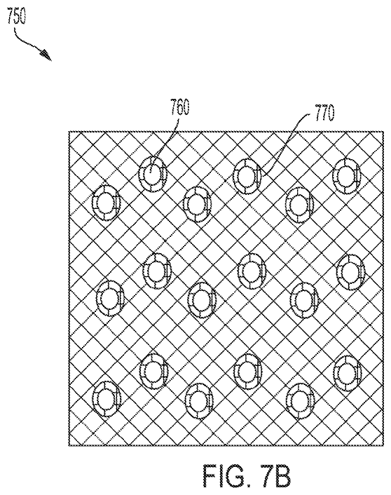

FIG. 7A provides a flow diagram of a method for producing a laminate including the water-responsive film that can be used in an absorbent article, according to some embodiments. In some embodiments, a method 700 of producing a laminate is provided. The method 700 may include providing a film 710. The film includes a water-soluble polymer and an aromatic TPU. As described herein, the water-soluble polymer may be substantially crystalline. For example, the water-soluble polymer may have a crystallinity of greater than 50%, e.g., greater than 55%, greater than 60%, greater than 65%, greater than 70%, greater than 75%, greater than 80%, greater than 85%, or greater than 90%. In some embodiments, the water-soluble polymer may comprise polyethylene oxide, hydroxypropyl cellulose, polyvinyl alcohol, polyvinyl pyrrolidone, polyvinyl pyridine, gelatinized starch, nylon copolymers, polyacrylic acid, and mixtures thereof. In some embodiments, the aromatic TPU can have a tensile strength ranging from 10 MPa to 50 MPa. In some embodiments, the aromatic TPU can have a tensile modulus ranging from 1 MPa to 5 MPa.

The method 700 may include bonding a portion of the film to a substrate 720. For example, the film may be adhesively bonded to at least one substrate to produce a laminate (e.g., a liner). The film can be bonded to at least one substrate (top and/or bottom) to control lateral expansion and closing of the apertures that function as a valve. In some embodiments, the film is bonded to any of the layers of an absorbent article. For example, the film can be bonded to one or more of a fluid transfer layer, an absorbent body, or a backsheet.

In some embodiments, the method 700 may include cutting or scoring the laminate 730. In some embodiments, the laminate is stamped, scored, or cut to include one or more apertures. For example, the laminate can be laser-cut to include any of the valve designs described in FIGS. 2A-4F. In some embodiments, the film can be cut before bonding the film to a substrate. In some embodiments, the film and substrate are bonded to produce the laminate and the laminate can be cut or stamped to include one or more apertures.

In some embodiments, the laminate can be patterned to leave a specific unbound area adjacent to the one or more apertures of the film. For example, FIG. 7B shows an embodiment of the laminate 750 that includes a plurality of apertures 760. The region surrounding the apertures 760 can be the unbound region 770 of the film. For example, the unbound region 770 is not adhesively bonded to the substrate. The unbound region 770 of the film allows for lateral expansion of the film to close the apertures when contacted by water. The amount of expansion (e.g., lateral expansion) may be influenced by the composition of the film, the ratio of water-soluble polymer to thermoplastic polyurethane, and the amount of fluid contacting the film.

### Examples

Two sample absorbent articles were tested to determine the water-responsive properties of each absorbent article. The two absorbent articles comprised a laminate including a control film and a prototype film. The prototype film comprised 70 wt. % POLYOX™ WSR N-80 (polyethylene oxide, a water-soluble polymer) and 30 wt. % WANTHANE® WHT-F170 (an aromatic thermoplastic polyurethane). The prototype film had a crystallinity of 56% as measured by DSC analysis. The prototype film was bonded to a hydrophobic spunbond nonwoven to produce a laminate. The laminate was cut to include a bulls-eye shaped valve design. The prototype absorbent article included a surge material and a core below the laminate, completing the construction of the prototype absorbent article. The core included fluff and superabsorbent material.

The control film comprised 100 wt. % WANTHANE WHT-F170. The control film was bonded to hydrophobic spunbond nonwoven to produce a laminate. The laminate was cut to include a bulls-eye shaped valve design. The control absorbent article included a surge material and a core below the laminate, completing the construction of the control absorbent article. The core included fluff and superabsorbent material.

Each of the absorbent articles were tested to determine water responsive properties of the films. The absorbent articles were tested using a modified flat intake test. The modified flat intake test included insulting the absorbent articles twice with 85 ml of a 0.9 wt. % aqueous solution of NaCl. The absorbent articles were subjected to the first insult of 85 ml solution for two minutes. The absorbent articles were then rewet on blotter papers (under pressure) for a duration of two minutes. After a waiting period of eight minutes, the absorbent articles were subjected to a second insult of 85 ml of solution, and after two minutes, the rewet measurement was repeated.

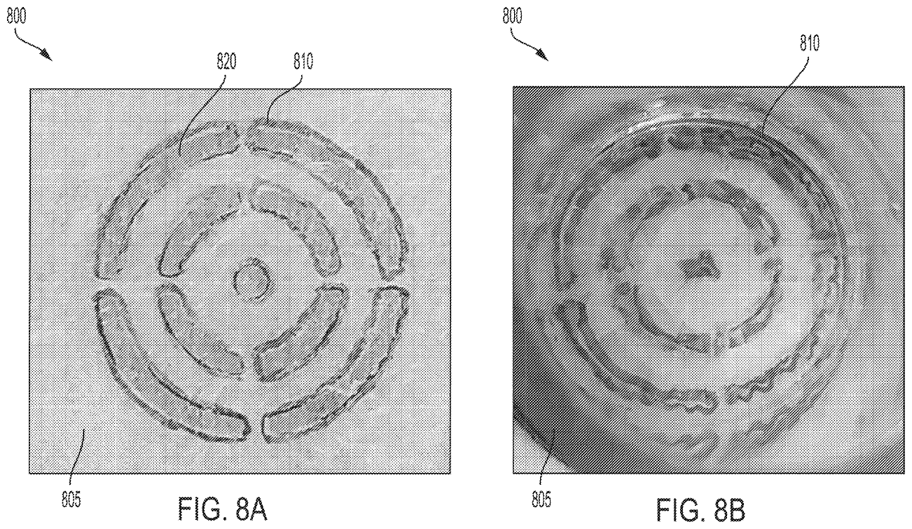

FIGS. 8A and 8B illustrate the expansion and retraction properties of the prototype absorbent article including a bulls-eye shaped pattern according to some embodiments of the present invention. FIGS. 8A and 8B provide two photographs showing the state of the prototype absorbent article 805 before insult 800 and after insult 850. The arcs outline the border between the film edge 810 of the laminate and the apertures 820. The photographs show that the space between the film edges 810 that define the aperture 820 of the bulls-eye design are clearly 1 mm or 2 mm apart prior to insult 800. However, following insult 850, the arcs have come closer together, indicating that when wet, the film of the laminate has expanded and closed the apertures 820 in the valve of the prototype absorbent article 805.

Table 3 shows the results of the modified intake and rewet test. Although there was little separation between the data for the prototype and control for the two intake times, the prototype with the film described herein showed an advantage over the control by substantially reducing rewet on both insults.

TABLE 3

| | 1^{st} Intake | 2^{nd} Intake | Rewet after | Rewet after |
|---|---|---|---|---|
| Sample | Time (s) | Time (s) | 1^{st} insult (g) | 2^{nd} insult (g) |
| Control | 21 | 36 | 8.5 | 14.2 |
| Prototype | 22 | 41 | 7.1 | 10 |

All patents, publications and abstracts cited above are incorporated herein by reference in their entireties. Various embodiments of the invention have been described in fulfillment of the various objectives of the invention. It should be recognized that these embodiments are merely illustrative of the principles of the present invention. Numerous modifications and adaptions thereof will be readily apparent to those skilled in the art without departing from the spirit and scope of the present invention as defined in the following claims.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto and the following embodiments:

### Embodiment 1

An absorbent article comprising a film comprising a water-soluble polymer and an aromatic thermoplastic polyurethane, wherein the film has a degree of crystallinity greater than or equal to 25%, wherein the film is configured to expand when in contact with a fluid.

### Embodiment 2

The absorbent article of any preceding or subsequent embodiment, wherein the water-soluble polymer comprises polyethylene oxide, hydroxypropyl cellulose, polyvinyl alcohol, polyvinyl pyrrolidone, polyvinyl pyridine, gelatinized starch, nylon copolymers, polyacrylic acid, or combinations thereof.

### Embodiment 3

The absorbent article of any preceding or subsequent embodiment, wherein the water-soluble polymer comprises polyethylene oxide, hydroxypropyl cellulose, polyvinyl alcohol, polyvinyl pyrrolidone, polyvinyl pyridine, gelatinized starch, nylon copolymers, polyacrylic acid, or combinations thereof.

### Embodiment 4

The absorbent article of any preceding or subsequent embodiment, wherein the film includes a patterned surface including one or more apertures, wherein the apertures comprise at least 10% of the total surface area of the film.

### Embodiment 5

The absorbent article of any preceding or subsequent embodiment, wherein a region adjacent the one or more apertures is treated with a hydrophilic agent.

### Embodiment 6

The absorbent article of any preceding or subsequent embodiment, wherein a portion of the film is bonded to a substrate.

### Embodiment 7

The absorbent article of any preceding or subsequent embodiment, wherein the region of the film adjacent the one or more apertures is not bonded to a substrate.

### Embodiment 8

The absorbent article of any preceding or subsequent embodiment, wherein the aromatic thermoplastic polyurethane comprises a polyester-based thermoplastic polyurethane.

### Embodiment 9

The absorbent article of any preceding or subsequent embodiment, wherein the film has a degree of crystallinity from 25% to 60%.

### Embodiment 10

The absorbent article of any preceding or subsequent embodiment, wherein the film comprises the water-soluble polymer in an amount from 10 wt. % to 90 wt. %, based on the total weight of the film.

### Embodiment 11

The absorbent article of any preceding or subsequent embodiment, wherein the film comprises the aromatic thermoplastic polyurethane in an amount from 10 wt. % to 90 wt. %, based on the total weight of the film.

### Embodiment 12

The absorbent article of any preceding or subsequent embodiment, wherein a weight ratio of water-soluble polymer to the aromatic thermoplastic polyurethane is at least 1:1.

### Embodiment 13

The absorbent article of any preceding or subsequent embodiment, wherein the film comprises the water-soluble polymer in an amount from 30 wt. % to 70 wt. %, based on the total weight of the film, thermoplastic polyurethane in an amount from 30 wt. % to 60 wt. %, based on the total weight of the film, and a ratio of ratio of water-soluble polymer to aromatic thermoplastic polyurethane is at least 1.5:1.

### Embodiment 14

The absorbent article of any preceding or subsequent embodiment, wherein the film comprises one or more additives comprising surfactant, absorbents, antibiotics, or skin benefit agents.

### Embodiment 15

The absorbent article of any preceding or subsequent embodiment, wherein the one or more additives comprise a superabsorbent.

### Embodiment 16

The absorbent article of any preceding or subsequent embodiment, wherein the absorbent article includes a fluid transfer layer between the film and an absorbent body.

### Embodiment 17

The absorbent article of any preceding or subsequent embodiment, wherein the film further comprises a 3-D structure treated with a hydrophobic agent.

### Embodiment 18

An absorbent article comprising: a film comprising a water-soluble polymer and an aromatic thermoplastic polyurethane, wherein the film has a degree of crystallinity greater than or equal to 25%; a substrate bonded to the film to produce a laminate; a backsheet; and an absorbent body between the laminate and the backsheet.

### Embodiment 19

A method of producing a laminate, the method comprising: providing a film, wherein the film comprises a water-soluble polymer and an aromatic thermoplastic polyurethane, wherein the film has a degree of crystallinity greater than 25%; and bonding a portion of the film to a substrate to produce a laminate.

### Embodiment 20

The method of any preceding or subsequent embodiment, wherein the film is configured to expand when in contact with body fluid.

### Embodiment 21

The method of any preceding or subsequent embodiment, further comprising cutting or scoring a pattern in the laminate including a plurality of apertures, wherein a region of the film adjacent the apertures is not bonded to the substrate.

What is claimed is:

1. An absorbent article comprising:
a film comprising a water-soluble polymer and an aromatic thermoplastic polyurethane, wherein the film has a degree of crystallinity greater than or equal to 25%, wherein the film is configured to expand when in contact with a fluid.

2. The absorbent article of claim 1, wherein the water-soluble polymer comprises polyethylene oxide, hydroxypropyl cellulose, polyvinyl alcohol, polyvinyl pyrrolidone, polyvinyl pyridine, gelatinized starch, nylon copolymers, polyacrylic acid, or combinations thereof.

3. The absorbent article of claim 1, wherein the film includes a patterned surface including one or more apertures, wherein the apertures comprise at least 10% of the total surface area of the film.

4. The absorbent article of claim 3, wherein a region adjacent the one or more apertures is treated with a hydrophilic agent.

5. The absorbent article of claim 4, wherein a portion of the film is bonded to a substrate.

6. The absorbent article of claim 4, wherein the region of the film adjacent the one or more apertures is not bonded to a substrate.

7. The absorbent article of claim 1, wherein the aromatic thermoplastic polyurethane comprises a polyester-based thermoplastic polyurethane.

8. The absorbent article of claim 1, wherein the film has a degree of crystallinity from 25% to 60%.

9. The absorbent article of claim 1, wherein the film comprises the water-soluble polymer in an amount from 10 wt. % to 90 wt. %, based on the total weight of the film.

10. The absorbent article of claim 1, wherein the film comprises the aromatic thermoplastic polyurethane in an amount from 10 wt. % to 90 wt. %, based on the total weight of the film.

11. The absorbent article of claim 1, wherein a weight ratio of water-soluble polymer to the aromatic thermoplastic polyurethane is at least 1:1.

12. The absorbent article of claim 1, wherein the film comprises the water-soluble polymer in an amount from 30 wt. % to 70 wt. %, based on the total weight of the film, thermoplastic polyurethane in an amount from 30 wt. % to 60 wt. %, based on the total weight of the film, and a ratio of ratio of water-soluble polymer to aromatic thermoplastic polyurethane is at least 1.5:1.

13. The absorbent article of claim 1, wherein the film comprises one or more additives comprising surfactant, absorbents, antibiotics, or skin benefit agents.

14. The absorbent article of claim 13, wherein the one or more additives comprise a superabsorbent.

15. The absorbent article of claim 1, further comprising a fluid transfer layer between the film and an absorbent body.

16. The absorbent article of claim 1, wherein the film further comprises a 3-D structure treated with a hydrophobic agent.

17. An absorbent article comprising:
a film comprising a water-soluble polymer and an aromatic thermoplastic polyurethane, wherein the film has a degree of crystallinity greater than or equal to 25%;
a backsheet; and
an absorbent body between the film and the backsheet.

18. A method of producing a laminate, the method comprising:
providing a film, wherein the film comprises a water-soluble polymer and an aromatic thermoplastic polyurethane, wherein the film has a degree of crystallinity greater than 25%; and
bonding a portion of the film to a substrate to produce a laminate.

19. The method of claim 18, wherein the film is configured to expand when in contact with body fluid.

20. The method of claim 18, further comprising cutting or scoring a pattern in laminate including a plurality of apertures, wherein a region of the film adjacent the apertures is not bonded to the substrate.
